(19) United States
(12) Patent Application Publication
(10) Pub. No.: US 2005/0136093 A1
(43) Pub. Date: Jun. 23, 2005

(54) Implant for transport and release for pharmacologically active agents as well as a process for producing the same

(75) Inventor: Roman Denk, Weidenstetten (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Polyzenix GmbH

(21) Appl. No.: 11/023,928

(22) Filed: Dec. 28, 2004

Related U.S. Application Data
(63) Continuation of application No. PCT/EP03/07197, filed on Jul. 4, 2003.

(30) Foreign Application Priority Data
Jul. 5, 2002 (DE) 10230190.5

(51) Int. Cl. A61F 2/00

(52) U.S. Cl. 424/426

(57) ABSTRACT

The present invention relates to an implant comprising (a) a polymer of the general formula (I) as a matrix material of the implant, wherein n is from 2 to ∞, and R¹ to R⁶ are the same or different and represent a halogenalkoxy, alkoxy, alkylsulfonyl, dialkylamino or aryloxy group or a heterocycloalkyl or heteroaryl group having nitrogen as a heteroatom, and at least one halogen substituent; and (b) at least one pharmacologically active agent incorporated into the matrix material, a process for producing the same and the use of a mixture of a polymer having the formula (I) and a pharmacologically active agent for the preparation of a pharmaceutical composition in the form of an implant for the treatment of disorders caused by implantation of said implant in a patient.

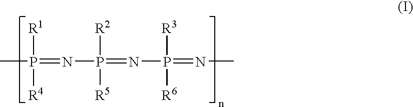

### CROSS-REFERENCE TO RELATED APPLICATIONS

This application is a continuation of co-pending International Application No. PCT/EP2003/007197, filed Jul. 4, 2003, entitled “Implant For Transport And Release For Pharmacologically Active Agents As Well As A Process For Producing The Same” which was published in the English language as WO 2004/004795 on Jan. 15, 2004, the entire disclosure of which as filed is incorporated herein by reference.

### BACKGROUND OF THE INVENTION

The present invention relates to an implant for transport and release of pharmacologically active agents as well as a process for producing the same.

The most serious complications caused by artificial implants are considered to be the increased deposition of thrombocytes on their exogenous surface. Further, the behaviour vis-à-vis bacteria, macrophages and proteins depositing on the surfaces, plays an important role, since these depositions mainly lead to irritations and reactions of the immune system during the growing in of implants. This is shown macroscopically, e.g., in the occurrence of inflammation reactions. In case of cardiovasculary stents, this leads to an increased cytogenesis and an increased growth of intima, resulting in a renewed blockage of a vessel. In general, this phenomenon is called as restenosis. In other devices this phenomenon leads to the necessity of an additional treatment with e.g. antibiotically active agents.

By applying bis-(trifluoroethoxy)polyphosphazene for the coating of implants of all kinds and catheters and other medically used devices, said afore-mentioned side reactions and inflammation reactions can be substantially suppressed. Further, particularly the hemocompatibility and thrombogenity of such surfaces is significantly improved. This material primarily has a passive protective function in such a way that native proteins are reversibly resorbed on said surfaces. However, should there be, in spite of the excellent biocompatibility of this layer, an undesired cell growth, this kind of surface treatment fails to offer an additional possibility to suppress said growth.

It turned out that, particularly in vasculary and cardiovasculary stents, the excellent properties of this material are suited to significantly improve the hemocompatibility as well as the biocompatibility of a steel or nitinol surface in vitro and in a rabbit test. The incorporation of medicaments into said layer is also known to pharmacologically ensure the prevention of restenosis.

In addition, other forms of surface treatment are used to improve the surface and the compatibility, in particular to cardiovasculary stents. In this context, it is referred to attempts for improving the surface by means of coatings consisting, e.g., of SiC, TiN, gold or other metals or hard materials or organic molecules, such as polymethacrylate or proteinaceous components, or cellular components, such as phosphoryl choline. Polyfluorinated variants of a set of materials have been also tested and analyzed.

In the past these coatings have turned out to be only of limited activity. Recent developments contain, in addition to a layer consisting e.g. of a biologically degradable component, which may e.g. consist of polylactide, a pharmacologically active component, such as rapamycin, taxol or other pharmacologically active substances. Other materials are also suited as a coating for pharmacologically active agents, but the possible fields of application of these materials are limited by the physical chemical properties of these compounds and the galenic formulation of the pharmacologically active substances.

Because of the extremely limited amount of a suited material (about 1 cm² total stent surface and a layer thickness of at most about 5 μm) such agent/carrier combinations will release the active agent only for a period of some weeks. This is due to the degradation of the coating layer, in case of biologically degradable layers, and the release of the active substances, which can only be provided in a limited amount. After releasing the agents, it is likely that side reactions due to the degradation of the coating layer occur.

In the prior art a variety of materials is known and has been analyzed, which may be used for the production of implants and are also suited as coating layers for pharmacologically active substances in the afore-mentioned limits. For example, in WO 98/56312 an expandable shell of e-PTFE is used as an implant. Pharmacologically active substances can be incorporated into this shell. Other materials for this use are materials which are cited in EP-A-0 810 845, U.S. Pat. No. 4,883,699 and U.S. Pat. No. 4,991,691. Additional polymers known for this purpose are for example hydrolyzed polyacrylonitrile (U.S. Pat. No. 4,480,642), hydrophilic polyether (U.S. Pat. No. 4,798,876), polyurethanediacrylate (U.S. Pat. No. 4,424,395); in addition, numerous hydrogels are known and may be used for this purpose. Potentially suitable materials may be extended by polyvinylpyrrolidone (PVP)-polymers, polyvinylalcohole (PVA), p(polyethylene oxide)-polymers (PEO) and polyhydroxyethyl methacrylate p(HEMA). Moreover, there are documents which describe the use of standard materials, such as polyurethane, polyethylene and polypropylene, as possible materials. Mixtures of these materials are also known. Additional materials are known from EP-A-0 804 909, which are suited as a coating for pharmacologically active agents. The properties of these compounds are different and it is considered that each of these materials is more or less suited as a coating material for pharmacologically active agents. For example, PVA is highyl soluble in liquids and thus, guarantees an extremely rapid release of the incorporated agent. However, in case of cardiovasculary stents, a rapid release of the agent is not desired, but to achieve a continuous, linear or constant activity over a long period of time.

Polyphosphazenes are a known class of compounds, which have been tested in the production of implants and in the improvement of the surface properties of such implants. From the literature several examples of polyphosphazenes are known and analyzed, having a very good biocompatibility, in particular hemocompatibility (DE 19613048). This property is also maintained when increasing the degree of fluorination. Moreover, the elasticity of such compounds is improved by incorporating higher fluorinated and longer side chains. However, an improvement of the biological properties or a therapeutic activity is not achieved by incorporating said side chains.

Moreover, several materials are known, wherein the side chain may be replaced by a pharmacologically active substance. These materials are, for example, cis-platinum derivatives, and other agents which are released by the degradation of the polymer. Fluoro derivatives are also known and analyzed (Allcock et al., Phosphorous and Nitrogen Compounds, Academic Press, 1972); in particular the trifluoroethoxy variant is well characterized with respect to their physical chemical properties. In the same manner the perfluorinated variants of these materials are known and analyzed. Although all of these compounds contribute to the improvement of the compatibility of implants and other medical devices in the body, these compounds are not ideal, since they have no biological activity which continues for a long period of time or they are degraded. Further, substitution degrees of several percent, usually more than 5%, as a minimum amount, are described in the literature to achieve the afore-mentioned effects (improvement of the elasticity). This is due to the branched main chain caused by a very strong tendency to hydrolysis and banching of the intermediates (poly-dichlorphosphazene). Thereby, only reduced substitution degrees may be achieved which are characterized by a relative high chlorine content (>0.05% of the final stage).

The polymer compound poly[bis(trifluoroethoxy)phosphazene], as a volume material, shows a good antithrombogenic activity (cf. Tur, “Untersuchungen zur Thrombenresistenz von Poly[bis(trifluorethoxy) phosphazen]” and Holleman Wiberg, “Stickstoff-Zusammensetzungen des Phosphors”, Lehrbuch der Anorganischen Chemie, 666-669, 91.-100. edition, Walter-de-Gruyter, 1985, and Tur, Vinogradova, “Entwicklungstendenzen bei polymeranalogen Umsetzungen von Polyphosphazen”, Acta Polymerica 39, 424-429, Nr. 8, 1988), and perfluorinated polyphosphazenes are described in DE 196 13 048 as a material for coating artificial implants, in particular for the coating of heart valves. However, these materials as described so far do not exhibit the required mechanical properties for the use as a matrix material or volume material (i.e. as the structure forming material) in combination with the required release rate of a pharmacologically active agent.

Thus, the technical problem underlying the present invention is to provide an implant which enables the safe transport and/or sustained release of a pharmacologically active agent (i.e. constant release of the active agent) into the body over a sufficient period of time, in particular into a vessel, and which is suited to sufficiently support the location of action, e.g., the vessel, mechanically and pharmacologically during the healing.

### BRIEF SUMMARY OF THE INVENTION

The solution to the above object is achieved by the embodiments as characterized in the claims.

In particular, the present invention provides an implant comprising:
(a) a polymer of the general formula (I) as a matrix material (or volume material) of the implant, 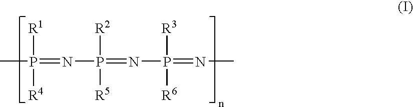
   wherein n is from 2 to ∞, preferably at least 10,000, more preferably 10,000 to 100,000, and most preferably 15,000 to 20,000, based on the repeating unit as defined in formula (I) above, and R¹ to R⁶ are the same or different and represent a halogenalkoxy, alkoxy, alkylsulfonyl, dialkylamino or aryloxy group or a heterocycloalkyl or heteroaryl group having nitrogen as a heteroatom, and at least one halogen substituent; and
(b) at least one pharmacologically active agent incorporated into the matrix material.

Surprisingly, the physical properties of polyphosphazenes and the disadvantages associated therewith are positively changed by admixing at least one pharmacologically active agent. That is, a mixtures of a specific polyphosphazene polymer, as defined above, with at least one pharmacologically active agent exhibits mechanical properties which enable the use of such a material as a matrix material for transport and/or sustained release of pharmalogically active agents and for mechanically supporting the location of action, such as vessels, if the implant is a stent, in a patient.

In this context, it is emphasized that the mixture of the above polymer having the formula (I) and the pharmacologically active agent is not used as a coating of an implant, but as a matrix or volume material which forms substantially the structure of the (whole) implant.

### DETAILED DESCRIPTION OF THE INVENTION

According to a preferred embodiment of the present invention, the implant consists of (a) a polymer of the general formula (I) as a matrix material, as defined herein, and (b) at least one pharmacologically active agent incorporated into the: matrix material, as defined below.

The terms “matrix material” and “volume material” as used herein relate to the structure forming material of the implant, for example a stent, which comprises the above polymer having the formula (I). Within the meaning of the present invention, a matrix material does not relate to a main constituent of a coating of an implant, but to the structure forming material itself.

The pharmacologically active agent may be incorporated in any form in the polymer having the formula (I) of the implant according to the present invention, for example, as a naked molecule or in nano- or micro-encapsulated form, so that the release of the agent may be regulated in a purposive manner. It is particularly preferred that the polymer (I) and the pharmacologically active agent form a homogenous and stable mixture to ensure a constant drug release profile over a long period of time.

The term “implant” encompasses any kind of suited implants, particulary implants which come into direct contact with tissue and/or body fluids of a patient. Examples of said implant are implants for e.g. breast, nose or ear, bone nails, bone screws, bone plates, artificial (urinary) bladder, artificial cartilage, dental implants, artificial bones for e.g. artificial hip or hip joints, artificial esophagus and artificial trachea; artificial (arterial and veinous) blood vessels; stents such as urological stents and cardiovascular stents; catheters such as urological catheters and cardiovascular catheters; cardiovascular grafts; emplastrums; dermatoplastics; seeds for the treatment of prostate hyperplasia; devices, e.g. in the gastrointestinal tract, in the prostata, in the urinary tract, or for the protection of neurons and neurofibers. Preferably the implant according to the present invention is in the form of a stent, in particular in the form of a cardiovasculary stent or an urological stent.

In the polymer of formula (I) it is preferable for at least one of the groups R¹ to R⁶ to be an alkoxy group substituted with at least one halogen atom such as a fluorine atom. In the polymer of formula (I), the alkyl groups in the halogenalkoxy, alkoxy, alkylsulfonyl and dialkylamino groups are, for example, straight-chain or branched-chain alkyl groups having 1 to 20 carbon atoms, wherein the alkyl groups can be substituted, for example, with at least one halogen atom, such as a fluorine atom.

Examples of (halogen)alkoxy groups are methoxy, ethoxy, propoxy and butoxy groups, which preferably can be substituted with at least one fluorine atom. The 2,2,2-trifluoroethoxy group is particularly preferred.

Examples of alkylsulfonyl groups are methylsulfonyl, ethylsulfonyl, propylsulfonyl and butylsulfonyl groups.

Examples of dialkylamino groups are dimethylamino, diethylamino, dipropylamino and dibutylamino groups.

The aryl group in the aryloxy group is, for instance, a compound having one or more aromatic ring systems, wherein the aryl group can be substituted, for instance, with at least one alkyl group as defined above.

Examples of aryloxy groups are phenoxy and naphthoxy groups and derivatives thereof.

The heterocycloalkyl group is, for example, a ring system having 3 to 7 atoms, at least one of the ring atoms being a nitrogen atom as a heteroatom. The heterocycloalkyl group can, for example, be substituted with at least one alkyl group as defined above. Examples of heterocycloalkyl groups are piperidinyl, piperazinyl, pyrrolidinyl and morpholinyl groups and their derivatives.

The heteroaryl group is, for example, a compound with one or more aromatic ring systems, wherein at least one ring atom is a nitrogen atom. The heteroaryl group can, for example, be substituted with at least one alkyl group as defined above. Examples of heteroaryl groups are pyrrolyl, pyridinyl, pyridinolyl, isoquinolinyl and quinolinyl groups and their derivatives.

The halogen substituent may be any appropriate halogen containing substituent within the meaning of the present invention, which is known to a person skilled in the art. According to a preferred embodiment the halogen substituent is a trifluoroethoxy group, preferably a 2,2,2-trifluoroethoxy group.

In a particularly preferred embodiment of the present invention, the polymer is poly[bis(triflubroethoxy)phosphazene] (PTFEP), represented by the following formula:
—[N═P(OCH₂CF₃)₂]ₘ—
wherein m is 2 to ∞, preferably at least 50,000 and most preferably 50,000 to 60,000, based on the above repeating unit.

The pharmacologically active agent to be used in the implant does not exhibit any specific limitations, and is preferably an organic (low or higher molecular weight) compound, especially an antimitogenic active agent such as a cytostatic (such as rapamycin, paclitaxel or taxol, respectively, etc.), a PDGF-inhibitor (such as tyrphostins etc.), a Raf-1 kinase inhibitor, a monoclonal antibody for integrin blockade of leukocytes, an antisense active agent (such as plasmid DNA, antisense-RNA etc.), superoxide dismutase, a radical trap (such as probucol etc.), a steroid, a statin (such as cerivastatin etc.), a corticosteroid (such as methotrexate, dexamethasone, methylprednisolan [sic] etc.), an adenylate cyclase inhibitor (such as forskolin etc.), a somatostatin analogue (such as angiopeptin etc.), an antithrombin agent (such as argatroban etc.), a nitric oxide donor, a glycoprotein IIb/IIIa receptor antagonist (such as urokinase derivatives, abciximab, tirofiban etc.), an antithrombotic agent (such as activated protein C, PEG-hirudin, prostaglandin analogues etc.), a vascular endothelial growth factor (VEGF), trapidil etc., and mixtures of these. In a preferred embodiment of the present invention, the pharmacologically active agent is tacrolimus, genexol, paclitaxel or taxol (cf. R. T. Liggins, W. L. Hunter and H. M. Burt, Journal of Pharmaceutical Sciences, Vol. 86, No. 12, 1997). By using said pharmacologically active agents (alone or in a mixture), a homogenous and stable mixture in the polymer having the formula (I), preferably in poly[bis(trifluoroethoxy)phosphazene], can be obtained.

It is desirable that the content of pharmacologically active agent(s) in the implant according to the present invention is as high as possible to e.g. prevent disorders caused by the implant such as restenosis, effectively. The weight ratio (mixing ratio) of the polymer having the formula (I) to the pharmacologically active agent(s) is preferably in a range of from about 50:1 to about 1:1, more preferably in a range of from about 10:1 to about 1:1, and most prefereably in a range of from about 5:1 to about 1:1. In this context, it is particularly preferred that the polymer (I) and the pharmacologically active agent(s) are miscible in each other and result in a homogenous and stable matrix material, and should preferably not result in a phase separation.

An implant in the form of a stent which is formed of poly[bis(trifluoroethoxy)-phosphazene] (PTFEP) as a matrix material and (the) pharmacologically active substance(s) incorporated therein is particularly preferred.

The release of the pharmacologically active agents is influenced by the specific combination of the polymer having the formula (I) and the pharmacologically active agent in a particular advantageous manner, so that the release rate of the pharmacologically active agent is significantly reduced and is suited to attain a constant release of the pharmacologically active agent over a sufficient period of time, such as several months, without damaging the surrounding cell tissue by a temporarily increased dosage.

The desired constant release rate of the pharmacologically active agent can be preferably achieved by a homogeneous and stable mixture of the polymer having the formula (I) and the at least one pharmacologically active agent. The “homogenous and stable mixture of the polymer having the formula (I) and the pharmacologically active agent” is achieved by adjusting suitable amounts of the polymer (I) and the pharmacologically active agent in such a way that no phase separation between the polymer (I) and the pharmacologically active agent will occur over a sufficient long period of time. An appropriate combination of a suitable amount of the polymer having the formula (I) and the suited pharmacologically active agent can be determined by a person skilled in the art.

Further, the elongation at break of the implant made of the above polymer having the formula (I) and a pharmacologically active agent is preferably at least 150%. Thus, the mixing ratio of the polymer having the formula (I) and the pharmacologically active agent is preferably adjusted to obtain an elongation at break of at least 150%.

The present invention further provides a process for producing an implant, as defined above, comprising the steps of:
(a) mixing the polymer of the general formula (I), as defined above, and at least one pharmacologically active agent, as defined above, and
(b) forming the mixture of step (a) into a desired form.

The step of mixing the polymer having the formula (I) and at least one pharmacologically active agent is performed in the molten state or in solution, e.g. in an appropriate solvent. Appropriate solvents for this process may be selected from polar aprotic solvents such as esters (such as ethyl acetate, propyl acetate, butyl acetate, ethyl propionate, ethyl butyrate etc.), ketones (such as acetone, ethyl methyl ketone etc.), amides (such as dimethylformamide etc.), sulfoxides (such as DMSO etc.) and sulfones (such as sulfolane etc.). Ethyl acetate is particularly preferred.

Premixing of the substances in solution which are then crystallized and compressed, is also possible.

The step of forming the mixture into a desired from of an implant (the final form), preferably a stent, is preferably performed by high-compression (high-compacting) or by melt-extrusion.

The implant according to the present invention can be used in the blood stream, in the lower urinary tract, in bones, for treating nerves and nerve fibers, in the gastrointestinal tract, on the skin and under the skin (subcutaneous), as breast implant, in the bladder and the prostrate, in arteries or veins. Further, the implant according to the present invention can be used for dental implants or bone implants, for example bone nails, bone plates and bone screws.

The present invention further provides the use of a mixture containing a polymer and a pharmacologically active agent, as defined above, for the preparation of a pharmaceutical composition (“a galenic formulation”) in the form of an implant for the treatment of disorders caused by implantation of said implant in a patient.

The subject matter of the present invention is further illustrated by way of the following examples without limitation thereto.

### EXAMPLES

The following examples 1 and 2, which relate to thick films, are given to simulate the implant according to the present invention, which is made of the above defined matrix material.

### Example 1

Polyzene®-F, which is poly[bis(trifluoroethoxy)phosphazene] (PTFEP), and a pharmacologically active agent are mixed in a particular mass ratio in an appropriate amount of pure ethyl acetate. For test purposes said mixture is formed into a solution cast film as described in the test method for measuring the elongation at break as described below.

The elongation at break (%) of the above matrix material (containing polymer having the formula (I) and a pharmacologically active agent) is measured in the form of a solution cast film (formed from the specific die-cast as described below) in accordance with the method described in “Starannikova, L. E., et al. Vysokomolekulyamye-Soedineniya, Ser. A & B, 36(11) (1994): 1,906”, as follows:

A die-cast made of stainless steel having the dimension 7×1×50 mm was filled with a solution of the polymer having the formula (I), a pharmacologically active agent and ethyl acetate in various concentrations and was dried. The length of said film was determined and the film was enlongated up to the moment of break. The percentage as defined in the unit “%” relates to the elongation at the moment of break with respect to the initial length of the above film.

The evaluation results with respect to the properties of the resulting film are summarized in Tables 1 and 2.

TABLE 1
(Taxol as active agent)

| Composition of the mixture | Elongation at break | Quality of the film |
|---|---|---|
| no active agent, 20 mg | 300% | homogenous |
| Polyzene ®=F, 1 ml | | |
| ethyl acetate | | |
| 2 mg Taxol, 18 mg | 220% | homogenous |
| Polyzene ®-F, 1 ml | | |
| ethyl acetate | | |
| 5 mg Taxol, 15 mg | 160% | homogenous |
| Polyzene ®-F, 1 ml | | |
| ethyl acetate | | |

TABLE 2
(Tacrolimus as active agent)

| Composition of the mixture | Elongation at break | Quality of the film |
|---|---|---|
| no active agent, 20 mg | 300% | homogenous |
| Polyzene ®-F, 1 ml | | |
| acetate | | |
| 2 mg Tacrolimus, 18 mg | 212% | homogenous |
| Polyzene ®-F, 1 ml | | |
| ethyl acetate | | |
| 5 mg Tacrolimus, 15 mg | 190% | homogenous |
| Polyzene ®-F, 1 ml | | |
| ethyl acetate | | |

### EXAMPLE 2

The taxol containing poly[bis(trifluoroethoxy)phosphazene] (PTFEP) films as shown in Table 3 were prepared on a substrate from a solution containing taxol, poly[bis(trifluoroethoxy)-phosphazene] and ethyl acetate by dip or spincoating. The film thickness was adjusted to about 0.3 to 1.0 μm. The resulting coating weight per surface unit is shown in Table 3.

In order to quantify the drug release properties of the samples, the increase of the drug concentration was measured by UV/vis-spectroscopy in a closed loop assembly in the course of one week.

TABLE 3

| PTFEP coating | taxol content | |
|---|---|---|
| (μg/cm²) | (μg/cm²) | drug release |
| 175 | about 75 | initial burst effect, constant |
| | | release after about 12 h |
| 150 | about 50 | initial burst effect, constant |
| | | release after about 12 h |
| 125 | about 42 | initial burst effect, constant |
| | | release after about 12 h |
| 75 | about 32 | initial burst effect, constant |
| | | release after about 12 h |

As can be taken from the above test results, after an initial burst effect (lasting approximately 12 h), taxol is released in a linear relationship. The respective release rates thereof which can be calculated by a simple calibration method are in a range of from about 0.1 to about 3.3 μg/ml per week, depending on the initial loading.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An implant comprising:
(a) a polymer of the general formula (I) as a matrix material of the implant, 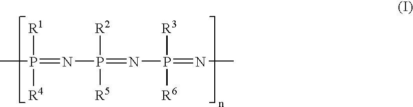
wherein n is from 2 to ∞, and R¹ to R⁶ are the same or different and represent a halogenalkoxy, alkoxy, alkylsulfonyl, dialkylamino or aryloxy group or a heterocycloalkyl or heteroaryl group having nitrogen as a heteroatom, and at least one halogen substituent; and
(b) at least one pharmacologically active agent incorporated into the matrix material.

2. The implant according to claim 1, which is in the form of a stent.

3. The implant according to claim 1, wherein the weight ratio of polymer (I) to the pharmacologically active agent is in a range of from 10:1 to 1:1.

4. The implant according to claim 1, wherein n in the polymer (I) is at least 10,000.

5. A process for producing an implant according to claim 1, comprising the steps of:
(a) mixing the polymer of the general formula (I), 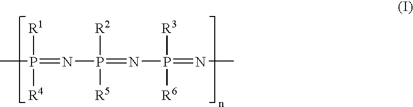
wherein n is from 2 to ∞, and R¹ to R⁶ are the same or different and represent a halogenalkoxy, alkoxy, alkylsulfonyl, dialkylamino or aryloxy group or a heterocycloalkyl or heteroaryl group having nitrogen as a heteroatom, and at least one halogen substituent; and at least one pharmacologically active agent, and
(b) forming the mixture of step (a) into a desired form.

6. The process according to claim 5, wherein the mixing is performed in the molten state or in solution.

7. The process according to claim 5, wherein the step of forming the mixture into a desired from is performed by high-compression or by melt extrusion.

8. The process according to claim 5, wherein the implant is formed into a stent.

9. Use of a mixture containing a polymer and a pharmacologically active agent as defined in claim 1 for the preparation of a pharmaceutical composition in the form of an implant for the treatment of disorders caused by implantation of said implant in a patient.
